(19) United States
(12) Patent Application Publication
(10) Pub. No.: US 2006/0173236 A1
(43) Pub. Date: Aug. 3, 2006

(54) Brachytherapy magazine seed indicator

(76) Inventors: Jack C. White, Alpharetta, GA (US); Matthew T. Groves, Lawrenceville, GA (US)

Correspondence Address: KNOBLE YOSHIDA & DUNLEAVY, LLC;EIGHT PENN CENTER, SUIT 1350, 1628 JOHN F. KENNEDY BOULEVARD, PHILADELPHIA, PA, 19103, US

(21) Appl. No.: 11/329,782

(22) Filed: Jan. 11, 2006

Related U.S. Application Data
(60) Provisional application No. 60/643,952, filed on Jan. 14, 2005.

(51) Int. Cl. A61M 36/00; A61N 5/00

(52) U.S. Cl. 600/7

(57) ABSTRACT

A indicator usable with plural brachytherapy element dispensing devices such as seed magazines or seed cartridges for determining the number of seeds remaining in the cartridge and/or the number of seeds that have been dispensed. In one embodiment, the indicator is releasably attachable to a brachytherapy-element dispensing device. In another embodiment, the indicator is merely associated with said brachytherapy element dispensing device. An indicating element having an upper surface is located within the indicator and is movable by engagement with a plunger of a seed magazine or cartridge. The indicating element can indicate the number of seeds remaining/dispensed in any number of ways, including movement relative to a gradated scale, via connection to a digital readout or via connection to an analog device. Also disclosed is an indicator which employs the position of at least a portion of a movable structure of said brachytherapy element dispensing device that moves as a result of dispensing of brachytherapy elements from said dispensing device, relative to said indicator to indicate the number of seeds remaining in, or dispensed from, the brachytherapy element dispensing device.

### CROSS-REFERENCE TO RELATED APPLICATIONS

This application claims the benefit of U.S. provisional patent application No. 60/643,952, filed on Jan. 14, 2005, pursuant to 35 U.S.C. § 119(e), the text of which is hereby incorporated by reference in its entirety.

### BACKGROUND OF THE INVENTION

1. Field of the Invention

The invention relates to the field of brachytherapy element dispensing devices. In particular, the invention relates to providing a device that, when associated with a brachytherapy seed and/or spacer magazine or cartridge, indicates the number of seeds and/or spacers remaining in the magazine or cartridge.

2. Description of the Related Technology

Brachytherapy is the treatment of medical conditions through the use of radioactive material placed at or near the affected area. Brachytherapy can either be applied externally or internally. One of the procedures used in Brachytherapy is the placement of radioactive seeds within a patient. Typically, a physician will implant several radioactive seeds in or around a tumor, thereby providing a radiation dose to the tumor.

Brachytherapy seeds for use in such procedures are frequently shipped in seed magazines suitable for loading the seeds into a needle or other seed implantation device. Mick® magazines are one example of a popular seed magazine for storage, shipping and dispensing of brachytherapy seeds. However, Mick® magazines, as well as some other seed magazines and seed cartridges, do not indicate the number of seeds that have been dispensed, or the number of seeds that remain in the magazine or cartridge. Consequently, unless the operator of the seed magazine keeps track of each seed dispensed, there is no way to determine the number of seeds remaining in the seed magazine or cartridge at any given time. Therefore, there exists a need to provide an efficient way to determine the number of seeds remaining in a brachytherapy seed magazine or cartridge and, consequently, determine the number of seeds that have already been dispensed.

Some devices that store and/or dispense brachytherapy seeds are provided with means for determining the number of seeds remaining in the device. For example, one device for determining the number of seeds in a brachytherapy seed-containing device can be found at, http://www.mentorcorp.com/cancer/prostatecancer/physician/PC_seedvue.htm. The SeedVue™ device has a transparent body that enables visualization of the seeds from any angle. The device additionally includes a seed counter. The seed counter is provided with graduated markings to facilitate determination of how many seeds remain in the device. It does not appear that the SeedVue™ seed indicator is removable or includes any means to augment its readability.

U.S. Pat. No. 6,572,527 to Steele, Sr. et al., discloses a seed holder with a means for determining the number of seeds remaining in the seed holder. The seed holder has graduated marks placed on an upper portion of a shaft of the seed holder. The graduated marks provide information about the number of seeds remaining in the holder. This device is difficult to use, however, because the small size of brachytherapy seeds requires that the graduated marks be closely spaced, thereby making it difficult to read the graduated marks and determine exactly how many seeds remain in the seed holder.

U.S. Pat. No. 6,508,755 to Ravins et al., discloses a seed applicator provided with a counter that uses a gear-type mechanism to count the seeds as they are dispensed. The counter is connected to the housing of the seed applicator.

U.S. Patent application publication no. 2002/0032360 to Fontayne et al., discloses a seed cartridge that has a counter. The counter is provided with a lens that permits the user to view the numeric markings indicating the number of seeds remaining in the seed cartridge.

U.S. Patent application publication no. 2004/0147800 to Barber et al., discloses a seed magazine with a gauge that moves into the housing of the seed magazine thereby indicating the number of seeds remaining in the seed magazine.

There remains a need in the field of brachytherapy seed magazines for a device with the ability to determine the number of seeds remaining in a seed cartridge or magazine and, consequently, the number of seeds that have already been dispensed from the magazine or cartridge.

There also remains a need for an improved device for indicating the number of seeds remaining in a seed cartridge or magazine.

### SUMMARY OF THE INVENTION

Accordingly, it is an object of certain embodiments of the invention to provide a device that, when associated with a brachytherapy element dispenser, can be used to determine the number of brachytherapy elements remaining in the dispenser or the number of brachytherapy elements that have been dispensed from the cartridge or magazine. One advantage of devices in accordance with the present invention is that a single indicator can be employed to provide information from a plurality of brachytherapy element dispensers.

According to a first aspect of the invention, there is provided an indicator for indicating a quantity of brachytherapy elements dispensed from, or located in, a brachytherapy element dispensing device. The indicator has a movable indicating element positioned to provide an indication responsive to a position of at least a portion of a portion of a structure of the brachytherapy element dispensing device that moves as a result of dispensing of one or more brachytherapy elements from said brachytherapy element dispensing device. There is also a readout operatively associated with the indicating element to indicate a number of brachytherapy elements dispensed from, or located in, the brachytherapy element dispensing device on an indication provided by the indicating element.

According to a second aspect of the invention, a method for indicating a number of brachytherapy elements located in, or dispensed from, a brachytherapy element dispensing device is disclosed. The method includes the step of positioning an indicator including an indicating element in an operative relationship with a movable structure of the brachytherapy element dispensing device that moves as a result of dispensing of brachytherapy elements from said dispensing device. The method also includes the steps of providing an indication responsive to a position of said movable structure of said brachytherapy element dispensing device, and providing a readout to indicate the number of brachytherapy elements located in, or dispensed from, said brachytherapy element dispensing device.

According to a third aspect of the invention, an indicator for indicating a quantity of brachytherapy elements dispensed from, or located in, a brachytherapy element dispensing device is provided. The indicator is associated with a brachytherapy element dispensing device and includes a readout operatively associated with at least a portion of a movable structure of the brachytherapy element dispensing device that moves as a result of dispensing of brachytherapy elements from said dispensing device, to thereby indicate a number of brachytherapy elements dispensed from, or located in, the brachytherapy element dispensing device based on a position of the portion of the movable structure relative to at least a portion of said indicator. The indicator is separable from said brachytherapy element dispensing device such that the indicator can be used to obtain information from plural brachytherapy element dispensing devices.

According to a fourth aspect of the invention, a method for indicating a number of brachytherapy elements located in, or dispensed from, a brachytherapy-element dispensing device is provided. The method includes the steps of positioning an indicator such that plural positions of at least a portion of a movable structure of the brachytherapy element dispensing device that moves as a result of dispensing of brachytherapy elements from said dispensing device, will provide indications by said indicator of a number of brachytherapy elements located in, or dispensed from, said brachytherapy element dispensing device, and determining a number of brachytherapy elements located in, or dispensed from said brachytherapy element dispensing device from said position of a portion of said movable structure relative to at least a portion of said indicator.

These and various other advantages and features of novelty that characterize the invention are pointed out with particularity in the claims annexed hereto and forming a part hereof. However, for a better understanding of the invention, its advantages, and the objects obtained by its use, reference should be made to the drawings which form a further part hereof, and to the accompanying descriptive matter, in which there is illustrated and described a preferred embodiment of the invention.

### BRIEF DESCRIPTION OF THE DRAWINGS

FIG. 1 shows a prior art seed magazine suitable for use with the present invention.

FIG. 2 shows a perspective view of a first embodiment of an indicator in accordance with the present invention.

FIG. 3 shows a partial cutaway end view of the indicator of FIG. 2.

FIG. 4 shows a perspective view of a second embodiment of an indicator in accordance with the present invention provided with a digital display.

FIG. 5 shows a perspective view of a third embodiment of an indicator in accordance with the present invention provided with an analog display.

FIG. 6 shows an alternative embodiment of a loaded prior art seed magazine suitable for use with the present invention.

FIG. 7 shows an alternative embodiment of an unloaded prior art seed magazine suitable for use with the present invention.

FIG. 8 shows a top down view of the magazine head of the prior art seed magazine of FIGS. 6-7 suitable for use with the present invention.

FIG. 9 shows a view of the seed magazine shown in FIGS. 6-8 provided with an indicator.

### DETAILED DESCRIPTION OF THE PREFERRED EMBODIMENT(S)

The term, “brachytherapy elements” includes brachytherapy seeds and brachytherapy spacers for use in providing spacing between brachytherapy seeds. Although the various embodiments of the invention described below are exemplified using brachytherapy seeds and brachytherapy seed magazines, it will be appreciated by a skilled person that the present invention is also applicable to devices for dispensing brachytherapy spacers and/or devices for dispensing both brachytherapy seeds and brachytherapy spacers.

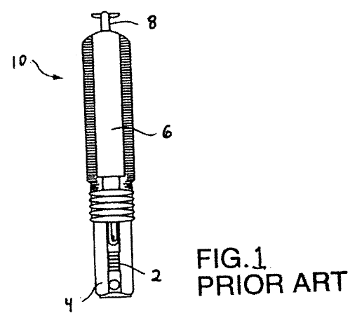

Referring now to the drawings, wherein like reference numerals designate corresponding structure throughout the several views, FIG. 1 shows a view of a Mick® brachytherapy seed magazine. Seed magazine 10 holds seeds 2 for use in brachytherapy inside seed cartridge 4. Magazine 10 also includes a magazine head 6. A dispensing actuator in the form of a spring-loaded plunger 8 is biased against the seeds 2 at the upper end of magazine 10 to facilitate seed movement into a needle chuck or other device to which the magazine 10 may be attached. Since plunger 8 is biased against seeds 2, plunger 8 moves downwardly relative to magazine head 6, each time a seed 2 is dispensed. Neither magazine 10 nor seed cartridge 4 includes a means for determining the number of seeds held in seed cartridge 4 at a given time. The inability to automatically keep track of the number of seeds remaining in seed cartridge 4 increases the chances of error in the implementation of the treatment plan since the operator may lose track of the number of seeds dispensed and, as a result, dispense too many or too few seeds. The instant invention overcomes this problem by providing a scale that can be utilized with such seed magazines or seed cartridges to indicate the number of seeds remaining in the magazine or cartridge.

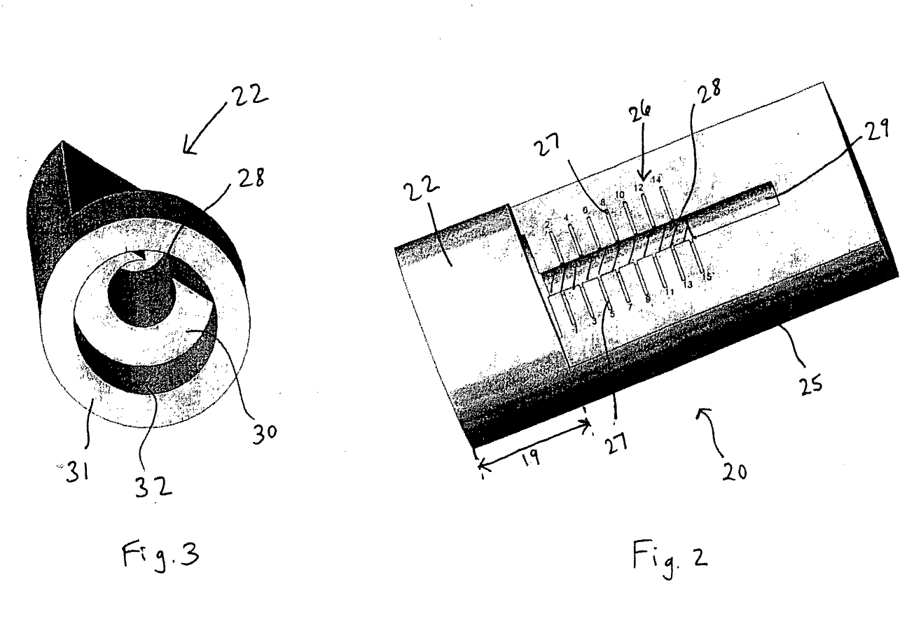

Referring now to FIG. 2, there is shown a perspective view of a first embodiment of an indicator 20 in accordance with the present invention. Indicator 20 includes a housing 25 that contains a mechanism for indicating the number of seeds remaining in a seed magazine or cartridge and/or the number of radioactive seeds remaining in a seed magazine or cartridge. At one end of indicator housing 25 is an attachment mechanism 22 shown in greater detail in FIG. 3. Indicator housing 25 is provided with a gradated scale 26 having gradations 27 that represent the number of seeds that have been dispensed from the seed magazine or cartridge 10. Indicator 20 also includes an indicating element 28 that is operatively connected to shoulder 30 of indicator 20, such that indicating element 28 moves relative to gradated scale 26 as shoulder 30 moves relative to indicator housing 25.

In this embodiment, indicating element 28 responds directly to the position of plunger 8 of seed magazine 10 shown in FIG. 1. FIG. 3 shows, in a partial cutaway view, attachment mechanism 22 of indicator 20 in greater detail. Attachment mechanism 22 includes an annular portion 31 that is sized to provide a friction fit between inner surface 32 and magazine head 6 of seed magazine 10.

Attachment mechanism 22 is adapted to releasably attach indicator 20 to a brachytherapy element dispensing device such that the indicator of the present invention can be employed to obtain information from plural brachytherapy element dispensing devices. Other ways of releasably attaching indicator 20 to seed magazine 10 via an attachment mechanism 22 are also possible. Exemplary means for attachment are a form fit, a snap-fit, a strap that encircles magazine head 6 and can be releasably closed, clasps, magnetic elements for engaging metallic seed magazines, an annular ring that opens to allow magazine head 6 to be placed within the annular ring, after which annular ring is closed to grasp magazine head 6, and other similar conventional devices. Attachment mechanism 22 is preferably located at one end of indicator 20, as shown in FIG. 2. The attachment of indicator 20 in this manner, allows the user to view the majority of seed magazine 10, even when indicator 20 is attached. This may facilitate data collection since a label on seed magazine 10 can be read during indication of the number of seeds 2 remaining in seed magazine 10.

Preferably, annular portion 31 of attachment mechanism 30 has a sufficient longitudinal thickness 19, as shown in FIG. 2, to permit attachment mechanism 22 to cover plunger 8 of seed magazine 10 for all possible positions of plunger 8. This will ensure that indicating elements 28 that are actuated by direct mechanical connection have sufficient space to move to all required locations to indicate the number of seeds dispensed or remaining in seed magazine 10. In addition, the provision of sufficient longitudinal thickness 19 also provides the advantage that plunger 8 cannot be inadvertently activated during use of indicator 20.

Indicator 20 also includes an indicating element 28 provided with an upper surface 30 as shown in FIG. 3. In use, indicator 20 is mounted onto the end of magazine head 6 from which plunger 8 of seed magazine 10 protrudes. Inner surface 32 frictionally engages the outer surface of magazine head 6 to retain indicator 20 on seed magazine 10 or otherwise engages the magazine in order to hold it in place (set screw, spring clip, adhesive, etc.). Indicating element 28 is moveable relative to housing 25 and attachment mechanism 22 of indicator. Thus, in use, upper surface 30 of indicating element 28 contacts the top of plunger 8 when indicator 20 is attached to a seed magazine 10 and positions indicating element 28 at a position that corresponds to the position of plunger 8. In a preferred embodiment, upper surface 30 of indicating element 28 engages plunger 8 in a fashion that prevents dislodgement of upper surface 30 from plunger 8 during typical use of magazine 10. Upper surface 30 of indicating element 28, or the interior of indicating element 28, can engage plunger 8 via a spring member, biasing member, a C-clip attachment, a frictional engagement, an adhesive or other suitable engagement means.

In alternative embodiments, plunger 8 or another movable structure of the brachytherapy element dispensing device may perform the role of indicating element 28, as described in greater detail below. In practice, any portion of the brachytherapy element dispensing device that moves as a result of dispensing of one or more brachytherapy elements from the dispensing device may perform the role of the indicating element 28. In an embodiment where plunger 8 performs the task of indicating the number of seeds remaining in seed magazine 10, the top portion of plunger 8 is positioned within indicator 20 so that plunger 8 is visible through lens 29. The movement of plunger 8 within indicator 20 will then signify the number of seeds within seed magazine 10.

Since plunger 8 is biased against seeds 2, the position of plunger 8 can be used as an indication of the number of seeds 2 remaining in seed magazine 10. Accordingly, indicating element 28 is displaced to a particular location by contact between upper surface 30 and plunger 8. The lower end of indicating element 28 is positioned a predetermined distance from upper surface 30 such that the lower end of indicating element 28 is positioned at the proper location relative to gradations 27 to indicate the correct number of brachytherapy seeds 2 remaining in seed magazine 10. Indicator 20 may optionally be provided with a stop 33, or other similar mechanism, to prevent movement of indicating element 28 beyond one end of gradated scale 26. At the other end of gradated scale 26, a surface can be provided within indicator housing 26 to prevent indicating element 28 from moving beyond the other end of gradated scale 26. Other conventional mechanisms for limiting the movement of indicating element 28 may also be employed in the indicator of the present invention.

Preferably, gradated scale 26 includes gradations 27 on both sides of indicating element 28 since this improves the readability of indicator 20 due to the relatively small spacing between gradations 27 that is required when direct mechanical actuation of indicating element 28 is employed. Specifically, due to the small size of seeds 2, plunger 8 will move only a small distance each time a seed 2 is dispensed from seed magazine 10. Hence, gradations 27 must be close together to reflect the small distance that plunger 8 moves when a seed 2 is dispensed.

In an optional embodiment, indicator 20 may be provided with a magnifying device such as a lens 29 positioned over indicating element 28 and gradated scale 26 for the purpose of magnifying gradations 27 and the position of the lower end of indicating element 28 to enhance the readability of indicator 20. Alternatively, indicating element 28 can include a mechanical device, such as a set of gears, not shown, for the purpose of causing lower end of indicating element 28 to move a greater distance in response to movement of plunger 8, to allow gradated scale 26 to be enlarged to improve readability. Other conventional means may be employed to achieve this mechanical adjustment.

In an alternative embodiment, gradated scale 26 can be reversed in order to indicate the number of seeds 2 that have been dispensed from seed magazine 10. In still another embodiment, gradated scale 26 can include two sets of gradations 27, one to indicate the number of seeds 2 remaining in seed magazine 10, and another set of gradations 27 to indicate the number of seeds that have already been dispensed from seed magazine 10.

In the preferred embodiment, magazine scale housing 25 is constructed of plastic to ensure that the device is lightweight and disposable, however it is possible to construct magazine scale housing 25 out of metal, or some other material.

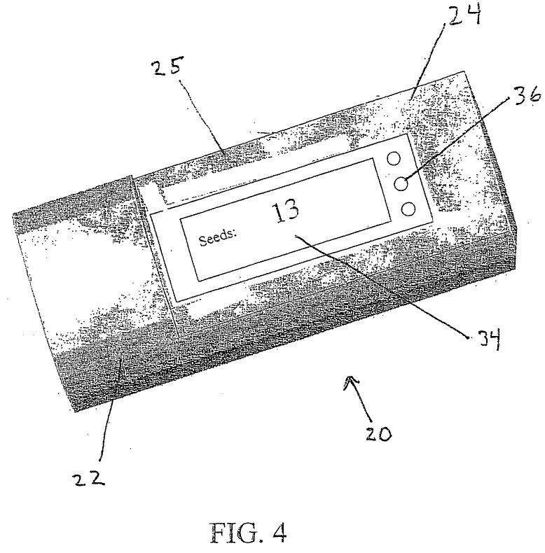

FIG. 4 shows a perspective view of an alternative embodiment of a magazine scale 20 provided with a digital display 34. In this embodiment, indicating element 28 is connected to a circuit that is responsive to the position of indicating element 28. Any conventional means for connecting the circuit to indicating element to provide a position-responsive output can be employed. An optional control 36 can be provided to allow the user to select whether to display the number of seeds remaining or the number of seeds that have been dispensed.

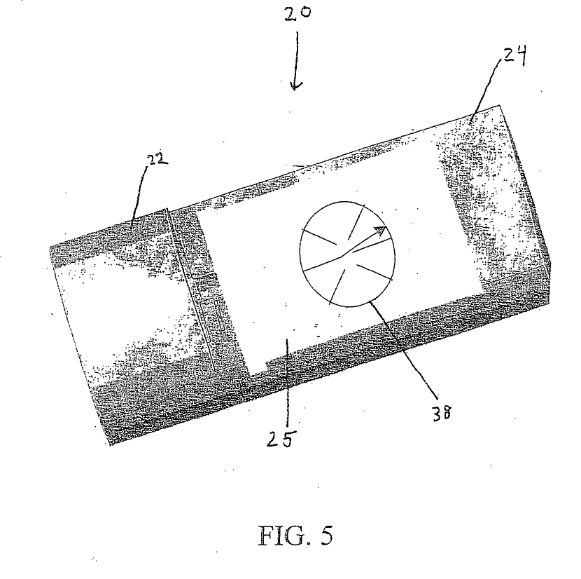

FIG. 5 shows a perspective view of an alternative embodiment of an indicator 20 provided with an analog readout 38, such as a dial. In this embodiment, indicating element 28 is connected to analog readout 38 that is responsive to the position of indicating element 28. Any conventional means for connecting the analog readout 38 to indicating element to provide a position-responsive output can be employed. In this embodiment, analog readout 38 may optionally be provided with two gradated scales, not shown, for indicating both the number seeds remaining in seed magazine 10, and the number of seeds dispensed.

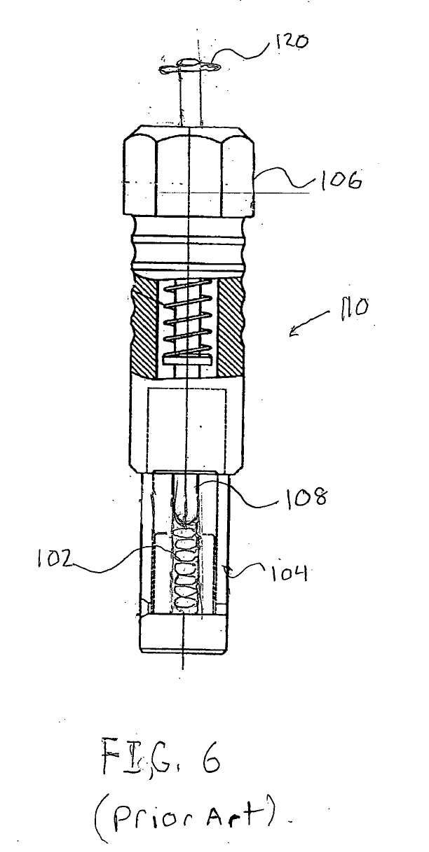

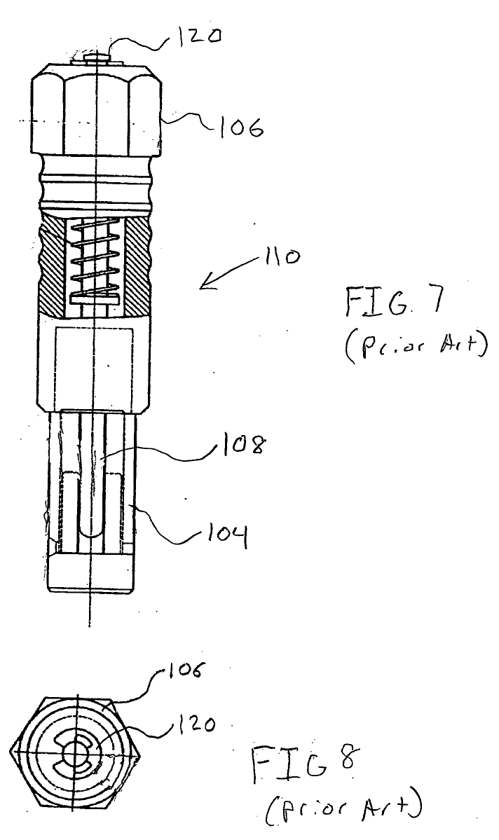

FIG. 6 shows an alternative embodiment of a prior art seed magazine suitable for use with the present invention. Seed magazine 110 holds seeds 102, and/or spacers, for use in brachytherapy, inside seed cartridge 104. Magazine 110 also includes a magazine head 106. A dispensing actuator in the form of a spring-loaded plunger 108 is biased against seeds 102 at the upper end of magazine 110 to facilitate seed movement into a needle chuck or other device to which magazine 110 may be attached. Since plunger 108 is biased against seeds 102, plunger 108 moves downwardly relative to magazine head 106, each time a seed 102 is dispensed. Seed cartridge 104 is adapted by size and configuration to receive a predetermined number of seeds 102 and/or spacers, which are biased by plunger 108 in a downward direction towards the distal end of seed cartridge 104. Clip member 120 is located at the distal end of magazine head 106 and is attached to plunger 108. Clip member 120 maintains plunger 108 in engagement with magazine head 106 and seed cartridge 104 when plunger 108 is fully depressed, as shown in FIG. 7.

FIG. 7 shows magazine 110 after seeds 102 have been dispensed. After seeds 102 have been dispensed, clip member 120 engages magazine head 106 and prevents further movement of plunger 108. Neither the prior art magazine 110, nor the prior art seed cartridge 104 includes a means for determining the number of seeds held in seed cartridge 104 at a given time.

FIG. 8 shows a top view of magazine head 106. Clip member 120 is shown engaged with the distal end of magazine head 106. When utilizing indicator 20 and attachment mechanism 22 shown in FIGS. 2 and 3 respectively, attachment mechanism 22 engages clip member 120. Indicator 20 also includes an indicating element 28 provided with an upper surface 30 as shown in FIG. 3. In use, indicator 20 is mounted onto the end of magazine head 6 from which clip member 120 of seed magazine 110 protrudes. Inner surface 32 frictionally engages the outer surface of magazine head 106 to retain indicator 20 on seed magazine 110 or otherwise engages the magazine in order to hold indicator 20 in place (e.g. by a set screw, spring clip, adhesive, etc.). Indicating element 28 is moveable relative to housing 25 and attachment mechanism 22 of indicator. Thus, in use, upper surface 30 of indicating element 28 contacts the top of clip member 120 when indicator 20 is attached to a seed magazine 110 and positions indicating element 28 at a position that corresponds to the position of clip member 120. In a preferred embodiment, upper surface 30 of indicating element 28 engages clip member 120 in a fashion that prevents dislodgement of upper surface 30 from clip member 120 during typical use of magazine 110. Upper surface 30 of indicating element 28, or the interior of indicating element 28, can engage clip member 120 by, for example, use of a spring member, biasing member, a C-clip attachment, frictional engagement portion, or an adhesive.

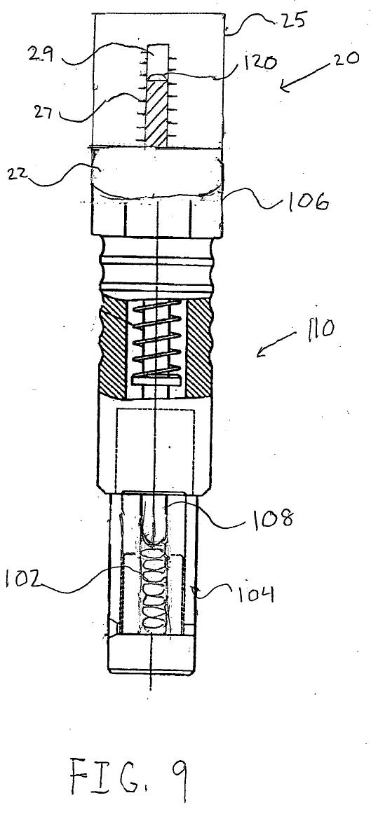

As shown in FIG. 9, in an alternative embodiment, clip member 120 may perform the role of indicating the number of seeds 102 remaining in magazine 110 thereby eliminating the need for an additional indicating element. In an embodiment where clip member 120 performs the task of indicating the number of seeds 102 remaining in seed magazine 110, the top portion of clip member 120 is positioned within indicator 20 so that clip member 120 is visible through window or lens 29. An attachment mechanism 22 may be employed to engage magazine head 106. The location of clip member 120 within indicator 20 will then indicate the number of seeds 102 within seed magazine 10, or, alternatively, the number of seeds 102 that have already been dispensed from seed magazine 10. In this embodiment, the scale of indicator 20 is adjusted to correspond to the position of clip member 120 when a given number of seeds 102 are within, or have been dispensed from, seed magazine 10 so that the clip member 120 provides the indication of the number of seeds 102 remaining in, or dispensed from, seed magazine 10.

One of the benefits of the present invention is that a single indicator can be employed to obtain information from a plurality of brachytherapy element dispensing devices and, as a result, the user need only have one indicator for this purpose. In this regard, the indicator of the present invention is separable from the brachytherapy element-dispensing device in order to provide this advantage. In one set of embodiments, described in detail above, the separability of the indicator is obtained by releasably attaching the indicator to the brachytherapy-element dispensing device such that the indicator can later be removed from the dispensing device.

In other embodiments of the present invention, the indicator need not include an attachment mechanism. In such embodiments, the structure of the indicator is such that it can be reliably associated with the brachytherapy element dispensing device, without attachment, in a manner whereby the indicator can provide an indication of the number of brachytherapy elements remaining in, or dispensed from the brachytherapy element dispensing device. These embodiments are particularly advantageous when using a portion of the brachytherapy element dispensing device as the indicating element since the indicator of the present invention can then be formed from a readout and a structure for associating the readout with the dispensing device in a position whereby the movable element of the dispensing device operates in conjunction with the readout to provide the desired indication. For example, a gradated scale can be positioned relative to a plunger of a dispensing device such that positions of the plunger correspond to indications of the number of brachytherapy elements on the gradated scale.

It is also possible to merely associate the indicator with the dispensing device when employing a special indicating element in the indicator. In such a case, the device will require some structure for translating a position of a movable portion of the dispensing device to an indication provided by said indicator. This can be a simple, mechanical interaction, e.g. a surface of the indicator contacts the plunger and moves when the plunger moves, or it can be an electrical, optical, or electromechanical interaction using any suitable conventional means.

It is also possible in some embodiments that the indicator include a radiation shielding material in order to protect a user of the device from radiation exposure. The entire indicator may be constructed of a shielding material, or alternatively, shielding material may be included in or on portions of the indicator in order to provide devices that can effectively shield a user of the device from radiation exposure. The geometry of the device and/or the geometry of the shielding material may also be customized to provide the desired shielding. By employing selective usage of the radiation shielding material on areas of the indicator where potential radiation exposure can occur, the overall cost of device construction can be reduced. The shielding material may also be retractable thereby enabling a user to employ the shielding material during potential exposure to radiation, and retracting the shielding material when exposure no longer poses a threat.

The shielding material can be a wide variety of materials, such as a high-density metal, e.g., lead or tungsten, or other materials (including lower density materials, such as plastics) that are effective in blocking radiation. The shielding material can also be comprised of more than one radiation shielding material.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

What is claimed is:

1. An indicator for indicating a quantity of brachytherapy elements dispensed from, or located in, a brachytherapy element dispensing device, said indicator comprising:
an indicating element responsive to a position of a movable structure of said brachytherapy element dispensing device that moves as a result of dispensing of brachytherapy elements from said dispensing device;
a structure for operatively associating said indicating element with said movable structure such that movement of said movable structure causes a corresponding change in an indication provided by said indicating element, and
a readout operatively associated with said indicating element to indicate a number of brachytherapy elements dispensed from, or located in, said brachytherapy element dispensing device based on an indication provided by said indicating element, and
wherein said indicator is separable from said brachytherapy element dispensing device to allow use of said indicator with a plurality of said brachytherapy element dispensing devices.

2. The indicator of claim 1, wherein said indicating element is a mechanical device including a portion that is positioned relative to said readout to indicate the number of brachytherapy elements dispensed from, or located in, said brachytherapy element dispensing device.

3. The indicator of claim 2, wherein said structure for operatively associating said indicating element with said movable structure comprises a mechanical connection for causing said indicating element to move a distance that is greater than a distance moved by said movable structure.

4. The indicator of claim 3, wherein said mechanical connection comprises a gear.

5. The indicator of claim 2, wherein said readout comprises gradations indicating a number of brachytherapy elements remaining in said brachytherapy element dispensing device.

6. The indicator of claim 2, wherein said readout comprises gradations indicating a number of brachytherapy elements that have been dispensed from said brachytherapy element dispensing device.

7. The indicator of claim 1, wherein said readout is digital.

8. The indicator of claim 1, wherein said readout is analog.

9. The indicator of claim 1, further comprising an attachment mechanism for attaching said indicator to said brachytherapy element dispensing device, and said attachment mechanism comprises a surface adapted to provide a friction fit with said brachytherapy element dispensing device.

10. The indicator of claim 1, further comprising a magnification device for magnifying said readout.

11. The indicator of claim 1, further comprising a radiation shielding material.

12. A method for indicating a number of brachytherapy elements located in, or dispensed from, a brachytherapy element dispensing device, said method comprising the steps of:
positioning an indicator including an indicating element in an operative relationship with a movable structure of said brachytherapy element dispensing device that moves as a result of dispensing of brachytherapy elements from said dispensing device,
providing an indication responsive to a position of said movable structure of said brachytherapy element dispensing device; and
providing a readout to indicate the number of brachytherapy elements located in, or dispensed from, said brachytherapy element dispensing device.

13. A method in accordance with claim 12, wherein said positioning step comprises releasably attaching said indicator to said brachytherapy element dispensing device.

14. A method in accordance with claim 13, wherein said step of providing a readout comprises associating the indicating position of said indicating element with a gradated scale.

15. An indicator for indicating a quantity of brachytherapy elements dispensed from, or located in, a brachytherapy element dispensing device, said indicator comprising:
a readout, and
structure for operatively associating said readout with said brachytherapy element dispensing device such that a position of at least a portion of a movable structure of said brachytherapy element dispensing device that moves as a result of dispensing of brachytherapy elements from said dispensing device is associated with said indicator in a manner whereby said readout gives an indication of a quantity of brachytherapy elements dispensed from, or located in, a brachytherapy element dispensing device, based on a current position of said portion of said movable structure relative to a portion of said indicator, and
wherein said indicator is separable from said brachytherapy element dispensing device to allow use of said indicator with a plurality of said brachytherapy element dispensing devices.

16. The indicator of claim 15, wherein said readout comprises gradations indicating a number of brachytherapy elements remaining in said brachytherapy element dispensing device.

17. The indicator of claim 15, wherein said readout comprises gradations indicating a number of brachytherapy elements that have been dispensed from said brachytherapy element dispensing device.

18. The indicator of claim 15, wherein said readout is digital.

19. The indicator of claim 15, wherein said readout is analog.

20. The indicator of claim 15, wherein said indicator further comprises an attachment mechanism for releasably attaching said indicator to said brachytherapy element dispensing device, and said attachment mechanism comprises a surface adapted to provide a friction fit with said brachytherapy element dispensing device.

21. The indicator of claim 15, further comprising a magnification device for magnifying said readout.

22. A method for indicating a number of brachytherapy elements located in, or dispensed from, a brachytherapy element dispensing device, said method comprising the steps of:
positioning an indicator relative to said brachytherapy element dispensing device such that plural positions of at least a portion of a movable structure of said brachytherapy element dispensing device that moves as a result of dispensing of brachytherapy elements from said dispensing device, will provide an indication by said indicator of a number of brachytherapy elements located in, or dispensed from, said brachytherapy element dispensing device; and
determining a number of brachytherapy elements located in, or dispensed from, said brachytherapy element dispensing device from said position of said portion of said movable structure relative to said indicator.

23. A method in accordance with claim 22, wherein said positioning step comprises releasably attaching said indicator to said brachytherapy element dispensing device.

24. A method in accordance with claim 22, wherein said determining step comprises associating a position of said portion of said movable structure with a gradated scale that forms part of said indicator.
